(19) United States
(12) Patent Application Publication
(10) Pub. No.: US 2022/0249381 A1
(43) Pub. Date: Aug. 11, 2022

(54) COMPRESSED MACITENTAN COMPOSITIONS, METHODS AND USES THEREOF

(71) Applicant: TECNIMEDE - SOCIEDADE TÉCNICO-MEDICINAL, SA, Sintra (PT)

(72) Inventors: João Pedro SILVA SERRA, Sintra (PT); Ricardo José Camilo FERREIRA PEREIRA, Sintra (PT)

(21) Appl. No.: 17/622,305

(22) PCT Filed: Jul. 3, 2020
(86) PCT No.: PCT/IB2020/056304

§ 371 Date: Dec. 23, 2021

(30) Foreign Application Priority Data
Jul. 5, 2019 (PT) 115632
Jul. 8, 2019 (EP) 19185065.0

(51) Int. Cl. A61K 9/20; A61K 31/506; A61P 9/12

(52) U.S. Cl. CPC A61K 9/2018; A61K 31/506; A61K 9/2054; A61P 9/12; A61K 9/2013; A61K 9/2095; A61K 9/2059

(57) ABSTRACT

The present disclosure also relates to compressed compositions comprising a therapeutically effective amount of macitentan, a sugar alcohol diluent and a surfactant. It also relates to the process of obtaining said compressed compositions, oral pharmaceutical compositions comprising them, and their use in the treatment of pulmonary arterial hypertension.

The present disclosure also relates to solid oral pharmaceutical compositions comprising a therapeutically effective amount of macitentan that simultaneously display improved stability properties and adequate dissolution profiles represents an unmet need in the field of pharmaceutical macitentan formulation.

### TECHNICAL FIELD

The present invention relates to compressed compositions comprising a therapeutically effective amount of macitentan, a sugar alcohol diluent and a surfactant.

It also relates to the process of obtaining said compressed compositions, oral pharmaceutical compositions comprising them, and their use in the treatment of pulmonary arterial hypertension.

### BACKGROUND

Macitentan is the international non-proprietary name (INN) of the chemical compound N-[5-(4-Bromophenyl)-6-[2-[(5-bromo-2-pyrimidinyl)oxy]ethoxy]-4-pyrimidinyl]-N′propylsulfamide, also known by the code name ACT-064992. Its molecular formula is C₁₉H₂₀Br₂N₆O₄S and its molecular weight is 588.27 g·mol⁻¹. It has the following structural formula:

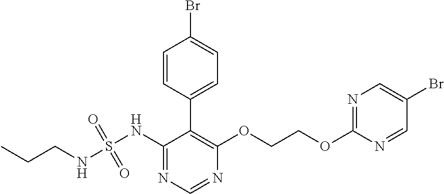 [SMILES: CCCNS(=O)(=O)Nc1ncnc(OCCOc2ncc(Br)cn2)c1-c1ccc(Br)cc1]

Macitentan is an endothelin receptor antagonist (ERA) currently approved and sold in Europe (EU and other countries), USA and Japan, under the trademark Opsumit, for the treatment of pulmonary arterial hypertension. Macitentan is a dual endothelin (ET) receptor antagonist that selectively inhibits the binding of ET-1 to ET_{A} and ET_{B} receptors. Macitentan inhibits the effects mediated by these receptors.

Macitentan is insoluble in aqueous solutions at room temperature at pH 1.2, 4, 6.8, 7, 9, and is only slightly soluble in organic solvents methanol and ethanol. It is a Biopharmaceutics Classification System (BCS) Class II compound, characterized by a high permeability and low solubility. Its solvation rate limits the bioavailabity exhibited in vivo.

MCT-IMA is the most prevalent impurity of macitentan formed either by oxidation or hydrolysis. It is also the major metabolite excreted in faeces. (Yerra, N. V. et al. Rapid Commun Mass Spectrum 2018, 15, 32, (13), 1075-1084; Thummar, M. et al. Chromatographia 2018, 81, 3, 525-531) (Bruderer, S. et al. Xenobiotica, 2012, 1-10).

Document WO 2002053557 A1 discloses macitentan and its preparation method.

Document WO 2007031933 and U.S. Pat. No. 8,367,685B2 discloses solid oral pharmaceutical compositions of macitentan. The results hint that, of all the possibilities tested, the compositions evidencing better behaviour were the ones comprising a high dosage of lactose or lactose monohydrate as filler.

Document IN 201641023754 discloses macitentan compositions comprising lactose as a filler.

Document WO 2014173805 discloses macitentan compositions comprising a specific polymorphic form. In particular, document WO 2014173805 discloses the preparation of a pharmaceutical composition comprising crystalline macitentan free base.

Document WO 2018153925 discloses macitentan compositions which does not contain surfactants.

Document IN 201641002749 discloses macitentan compositions obtained by extrusion method.

Document CN107913256 relates to macitentan-disintegrating tablet contained the following component ratio: macitentan-5-10%, a disintegrant 5-20%, the adhesive 0.5-20%, lubricant 0.5-5%, flavouring agent 2-5%, filler 65-75 mg.

Document CN109260163 A relates to a stable macitentan tablet composition, to provide a qualitative clinical medicine.

Document WO 2010054845 A1 relates to a method for the preparation of calcium carbonate tablets, which method results in tablets that have improved storage stability with respect to dissolution.

These facts are disclosed in order to illustrate the technical problem addressed by the present disclosure.

### General Description

In view of the drawbacks to the prior art, we hereby disclose solid oral pharmaceutical compositions comprising a therapeutically effective amount of macitentan that simultaneously display improved stability properties and adequate dissolution profiles.

This disclosure relates to compressed compositions comprising a therapeutically effective amount of macitentan, a sugar alcohol diluent and a surfactant. It also relates to the process of obtaining said compressed compositions, oral pharmaceutical compositions comprising them, and their use in the treatment of pulmonary arterial hypertension.

The present disclosure also relates to solid oral pharmaceutical compositions comprising a therapeutically effective amount of macitentan that simultaneously display improved stability properties and adequate dissolution profiles.

The present disclosure also relates to compressed composition comprising a therapeutically effective amount of macitentan; a sugar alcohol diluent selected from a list consisting of: maltitol, xylitol, mannitol or mixtures thereof; and a surfactant selected from a list consisting of: sodium dodecyl sulfate (SDS), polysorbate 80 (Tween 80), or a mixture thereof.

In one embodiment, the compressed composition comprises a therapeutically effective amount of macitentan, a sugar alcohol diluent and a surfactant.

In an embodiment, the surfactant refers to polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate) or SDS (sodium dodecyl sulfate). Preferably, the surfactant will be selected from polysorbate 80 (polyoxyethylene (20) sorbitan monooleate) or SDS (sodium dodecyl sulfate).

In an embodiment for better results, said surfactant is sodium dodecyl sulfate, polysorbate 80, or a mixture thereof.

In an embodiment, the sugar alcohol diluent refers to ethylene glycol, glycerol, erythritol, threitol, arabitol, xylitol, ribitol, mannitol, sorbitol, galactitol, frucitol, iditol, inositol, volemitol, isomalt, maltitol, lactitol, marotriol, maltotetratol or polyglycitol.

In an embodiment for better results, the sugar alcohol diluent is selected from maltitol, xylitol, mannitol, or mixture thereof.

In an embodiment for better results, the sugar alcohol diluent is maltitol, xylitol, or mixtures thereof.

In a preferred embodiment, the surfactant is sodium dodecyl sulfate (SDS).

In a preferred embodiment, the surfactant is polysorbate 80 (Tween 80).

In one embodiment, the compressed composition of the present disclosure may further comprise at least one excipient selected from a non-sugar alcohol diluent, a disintegrant, a binder and a lubricant, preferably the non-sugar alcohol diluent is microcrystalline cellulose.

In a preferred embodiment, the compressed composition of the present disclosure may comprise from 0.1% to 15% (wt_{surfactant}/wtₜₒₜₐₗ) of surfactant, preferably from 0.5% to 10% (wt_{surfactant}/wtₜₒₜₐₗ), more preferably from 0.5% to 5% (wt_{surfactant}/wtₜₒₜₐₗ); even more preferably from 0.5 to 2% (wt_{surfactant}/wtₜₒₜₐₗ) of surfactant.

In a preferred embodiment, the compressed composition of the present disclosure may comprise from 0.9% to 10% (wt_{surfactant}/wtₜₒₜₐₗ) of surfactant, preferably from 2% to 10% (wt_{surfactant}/wtₜₒₜₐₗ), more preferably from 2% to 5% (wt_{surfactant}/wtₜₒₜₐₗ).

In a preferred embodiment, the compressed composition of the present disclosure may comprise:

maltitol as sugar alcohol diluent and sodium dodecyl sulfate as surfactant; or

maltitol as sugar alcohol diluent and polysorbate 80 as surfactant;

xylitol as sugar alcohol diluent and sodium dodecyl sulfate as surfactant; or

xylitol as sugar alcohol diluent and polysorbate 80 as surfactant;

mannitol as sugar alcohol diluent and sodium dodecyl sulfate as surfactant; or

mannitol as sugar alcohol diluent and polysorbate 80 as surfactant.

In a preferred embodiment, the compressed composition of the present disclosure may comprise from 15% to 85% (wt_{sugar alcohol diluent}/wtₜₒₜₐₗ) of sugar alcohol diluent, preferably from 25% to 75% (wt_{sugar alcohol diluent}/wtₜₒₜₐₗ), more preferably from 35% to 65% (wt_{sugar alcohol diluent}/wtₜₒₜₐₗ).

In a preferred embodiment, the compressed composition of the present disclosure may comprise from 1% to 45% (wt_{macitentan}/wtₜₒₜₐₗ) of macitentan, preferably from 5% to 35% (wt_{macitentan}/wtₜₒₜₐₗ), more preferably from 5% to 25% (wt_{macitentan}/wtₜₒₜₐₗ).

In a preferred embodiment, the compressed composition of the present disclosure may comprise from 5% to 65% (wt_{non-sugar alcohol diluent}/wtₜₒₜₐₗ) of non-sugar alcohol diluent, preferably from 10% to 50% (wt_{non-sugar alcohol diluent}/wtₜₒₜₐₗ), more preferably from 15% to 35% (wt_{non-sugar alcohol diluent}/wtₜₒₜₐₗ).

In one embodiment, the compressed composition comprises 5% to 25% (wt_{macitentan}/wtₜₒₜₐₗ), of macitentan, 35% to 65% (wt_{sugar alcohol diluent}/wtₜₒₜₐₗ), of sugar alcohol diluent and 0.5% to 5% (wt_{surfactant}/wtₜₒₜₐₗ), of surfactant.

In one embodiment, the compressed composition further comprises at least one excipient selected from a non-sugar alcohol diluent, a disintegrant, a binder and a lubricant.

In one embodiment the non-sugar alcohol diluent is microcrystalline cellulose, the disintegrant is crospovidone, the binder is povidone and the lubricant is magnesium stearate.

In one embodiment the non-sugar alcohol diluent is microcrystalline cellulose, the disintegrant is sodium starch glycolate, the binder is povidone and the lubricant is magnesium stearate.

One aspect of the present disclosure relates to compressed compositions prepared by wet granulation.

One aspect of the present disclosure relates to a compressed composition for use in the treatment of pulmonary arterial hypertension.

One aspect of the present disclosure relates to solid oral pharmaceutical compositions comprising compressed macitentan compositions.

One aspect of the present disclosure relates to solid oral pharmaceutical compositions comprising a compressed macitentan composition for use in the treatment of pulmonary arterial hypertension.

Another aspect of the present disclosure relates to a process for preparing a compressed composition comprising:
mixing macitentan, a sugar alcohol diluent, and optionally one or more excipients selected from a non-sugar alcohol diluent, a disintegrant, a binder, a glidant;
granulating the mixture obtained in the previous step with an aqueous solution of a surfactant wherein the surfactant is sodium dodecyl sulfate, or polysorbate 80, or combinations thereof;
lubricating the granulate obtained in the previous step with magnesium stearate; compressing the lubricated granulate obtained in the previous step.

Another aspect of the present disclosure relates to a solid oral pharmaceutical composition comprising a compressed composition prepared according to a process comprising:
mixing macitentan, a sugar alcohol diluent, and optionally one or more excipients selected from a non-sugar alcohol diluent, a disintegrant, a binder, a glidant;
granulating the mixture obtained in the previous step with an aqueous solution of a surfactant wherein the surfactant is sodium dodecyl sulfate, or polysorbate 80, or combinations thereof;
lubricating the granulate obtained in the previous step with magnesium stearate; compressing the lubricated granulate obtained in the previous step.

Another aspect of the present disclosure relates to a solid oral pharmaceutical composition for use in the treatment of pulmonary arterial hypertension comprising a compressed composition prepared according to a process comprising:
mixing macitentan, a sugar alcohol diluent, and optionally one or more excipients selected from a non-sugar alcohol diluent, a disintegrant, a binder, a glidant;
granulating the mixture obtained in the previous step with an aqueous solution of a surfactant wherein the surfactant is sodium dodecyl sulfate, or polysorbate 80, or combinations thereof;
lubricating the granulate obtained in the previous step, preferably with magnesium stearate;
compressing the lubricated granulate obtained in the previous step.

In an embodiment, the process for preparing a compressed composition, or a solid oral pharmaceutical composition for use in the treatment of pulmonary arterial hypertension wherein the sugar alcohol diluent is maltitol, xylitol, or mannitol.

In an embodiment, the process for preparing a compressed composition a solid oral pharmaceutical composition for use in the treatment of pulmonary arterial hypertension wherein the non-sugar alcohol diluent is microcrystalline cellulose.

In an embodiment, the process for preparing a compressed composition a solid oral pharmaceutical composition for use in the treatment of pulmonary arterial hypertension wherein the disintegrant is crospovidone or sodium starch glycolate or mixture thereof.

The process for preparing a compressed composition a solid oral pharmaceutical composition for use in the treatment of pulmonary arterial hypertension wherein the binder is povidone, copovidone, or mixture thereof.

In an embodiment, the non-sugar alcohol diluent refers to microcrystalline cellulose.

In an embodiment, the disintegrant refers to sodium starch glycolate, or crospovidone, or mixture thereof.

In an embodiment, the binder refers to povidone, copovidone, or mixture thereof. Preferably, the binder is povidone.

In an embodiment, the lubricant refers to calcium stearate, stearic acid, sodium stearyl fumarate or magnesium stearate, or mixture thereof. Preferably, the lubricant is magnesium stearate.

In an embodiment, the glidant refers to colloidal anhydrous silica or talc. Preferably, the glidant is anhydrous colloidal silica.

In an embodiment, the compressed compositions have been found to display improved stability and fast dissolution profile as compared to compressed compositions containing 10 mg of macitentan, lactose monohydrate, microcrystalline cellulose, sodium starch glycolate, povidone, magnesium stearate and polysorbate 80.

In the context of the present disclosure a compressed composition comprises any composition comprising macitentan and pharmaceutical excipients such as tablet cores, microtablets, caplets, beads, granulates and the like. Preferably, the compressed compositions are tablet cores.

In an embodiment, the solid oral pharmaceutical composition comprising a compressed macitentan composition is in the form of any solid oral composition intended for the pharmaceutical delivery of a pharmaceutical agent, preferably as tablets, microtablets, caplets, or as beads contained in capsules, or as granulates in sachets intended to provide individual dosage forms. Most preferably, the solid oral pharmaceutical compositions are in the form of tablets.

### BRIEF DESCRIPTION OF THE DRAWINGS

The following figures provide preferred embodiments for illustrating the description and should not be seen as limiting the scope of invention.

FIG. 1 illustrates the dissolution profile of the disclosed compositions (Examples 1, 2, 3 and 5) as compared to the dissolution profile of the reference product Opsumit®.

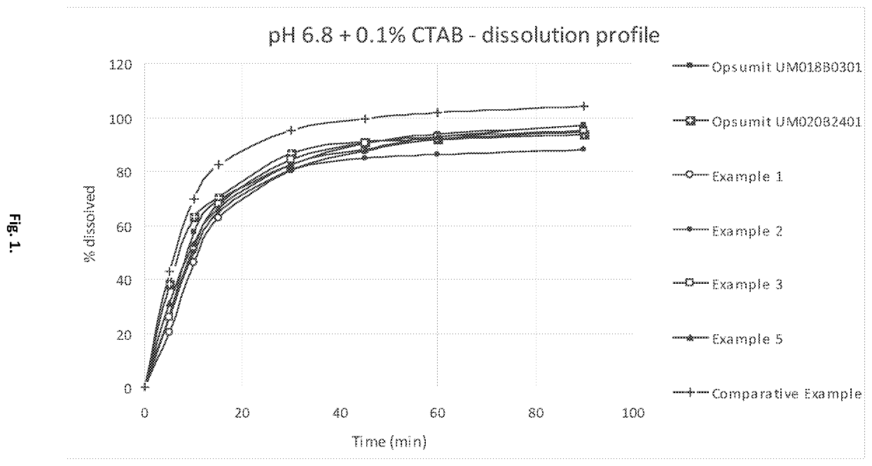

### DETAILED DESCRIPTION

The present invention relates to compressed compositions comprising a therapeutically effective amount of macitentan, a sugar alcohol diluent and a surfactant.

It also relates to the process of obtaining said compressed compositions, oral pharmaceutical compositions comprising them, and their use in the treatment of pulmonary arterial hypertension.

The present disclosure relates to solid oral pharmaceutical compositions comprising a therapeutically effective amount of macitentan that simultaneously display improved stability properties and adequate dissolution profiles.

In an embodiment, a compressed macitentan composition (Example 1, see in table 1 the composition detail) is prepared according to the following process:
a) Macitentan, xylitol, povidone, microcrystalline cellulose, silica colloidal anhydrous, and sodium starch glycolate powders are mixed in appropriate equipment.
b) The mixture is granulated with a granulation solution prepared with SDS and water.
c) The granulate is dried at 40° C. and calibrated with a sieve aperture of a 1 mm.
d) The calibrated granulate is lubricated with magnesium stearate and thereafter compressed.

TABLE 1
Compressed macitentan composition of example1

| Component | Amount (mg) | % |
|---|---|---|
| Macitentan | 10.12 | 14.45 |
| Xylitol | 37.00 | 52.86 |
| Microcrystalline cellulose | 15.88 | 22.69 |
| Sodium starch glycolate | 2.10 | 3.00 |
| Povidone | 2.10 | 3.00 |
| SDS | 1.40 | 2.00 |
| Silica colloidal anhydrous | 0.70 | 1.00 |
| Magnesium stearate | 0.70 | 1.00 |
| Total | 70 | 100.00 |

In an embodiment, a compressed macitentan composition (Example 2, see in table 2 the composition detail) is prepared analogously to the composition of Example 1 by substituting xylitol for maltitol.

TABLE 2
Compressed macitentan composition of example 2

| Component | Amount (mg) | % |
|---|---|---|
| Macitentan | 10.12 | 14.45 |
| Maltitol | 37.00 | 52.86 |
| Microcrystalline cellulose | 15.88 | 22.69 |
| Sodium starch glycolate | 2.10 | 3.00 |
| Povidone | 2.10 | 3.00 |
| SDS | 1.40 | 2.00 |
| Silica colloidal anhydrous | 0.70 | 1.00 |
| Magnesium stearate | 0.70 | 1.00 |
| Total | 70 | 100.00 |

In an embodiment, a compressed macitentan composition (Example 3, see in table 3 the composition detail) is prepared according to the following process:
a) Macitentan, xylitol, povidone, microcrystalline cellulose, and sodium starch glycolate powders are mixed in appropriate equipment.
b) The mixture is granulated with a granulation solution prepared with polysorbate 80 and water.
c) The granulate is dried at 40° C. and calibrated with a sieve aperture of a 1 mm.
d) The calibrated granulate is lubricated with magnesium stearate and thereafter compressed.

TABLE 3
Compressed macitentan composition of example 3

| Component | Amount (mg) | % |
|---|---|---|
| Macitentan | 10.12 | 14.45 |
| Xylitol | 37.00 | 52.86 |
| Microcrystalline cellulose | 16.58 | 23.69 |
| Sodium starch glycolate | 2.10 | 3.00 |
| Povidone | 2.10 | 3.00 |
| Polysorbate 80 | 1.40 | 2.00 |
| Magnesium stearate | 0.70 | 1.00 |
| Total | 70 | 100.00 |

In an embodiment, a compressed macitentan composition (Example 4, see in table 4 the composition detail) is prepared analogously to the composition of Example 3 by substituting xylitol for maltitol.

TABLE 4
Compressed macitentan composition of example 4

| Component | Amount (mg) | % |
|---|---|---|
| Macitentan | 10.12 | 14.45 |
| Maltitol | 37.00 | 52.86 |
| Microcrystalline cellulose | 16.58 | 23.69 |
| Sodium starch glycolate | 2.10 | 3.00 |
| Povidone | 2.10 | 3.00 |
| Polysorbate 80 | 1.40 | 2.00 |
| Magnesium stearate | 0.70 | 1.00 |
| Total | 70 | 100.00 |

In an embodiment, a compressed macitentan composition (Example 5, see in table 5 the composition detail) is prepared analogously to composition of Example 1 by substituting xylitol for mannitol.

TABLE 5
Compressed macitentan composition of example 5

| Component | Amount (mg) | % |
|---|---|---|
| Macitentan | 10.12 | 14.45 |
| Mannitol | 37.00 | 52.86 |
| Microcrystalline cellulose | 15.88 | 22.69 |
| Sodium starch glycolate | 2.10 | 3.00 |
| Povidone | 2.10 | 3.00 |
| SDS | 1.40 | 2.00 |
| Silica colloidal anhydrous | 0.70 | 1.00 |
| Magnesium stearate | 0.70 | 1.00 |
| Total | 70 | 100.00 |

In an embodiment, a compressed macitentan composition (Example 6, see in table 6 the composition detail) is prepared analogously to composition of Example 3.

TABLE 6
Compressed macitentan composition of example 6

| Component | Amount (mg) | % |
|---|---|---|
| Macitentan | 10.00 | 14.29 |
| Mannitol | 37.00 | 52.86 |
| Microcrystalline cellulose | 16.42 | 23.46 |
| Crospovidone | 3.50 | 5.00 |
| Povidone | 1.75 | 2.50 |
| Tween 80 | 0.63 | 0.90 |
| Magnesium stearate | 0.70 | 1.00 |
| Total | 70 | 100.00 |

In an embodiment, a compressed macitentan composition (Example 7, see in table 7 the composition detail) is prepared analogously to composition of Example 3.

TABLE 7
Compressed macitentan composition of example 7

| Component | Amount (mg) | % |
|---|---|---|
| Macitentan | 10.12 | 14.45 |
| Mannitol | 37.00 | 52.86 |
| Microcrystalline cellulose | 16.58 | 23.69 |
| Sodium starch glycolate | 2.10 | 3.00 |
| Povidone | 2.10 | 3.00 |
| Tween 80 | 1.40 | 2.00 |
| Magnesium stearate | 0.70 | 1.00 |
| Total | 70 | 100.00 |

In an embodiment, a compressed macitentan composition (Example 8, see in table 8 the composition detail) is prepared analogously to composition of Example 3.

TABLE 8
Compressed macitentan composition of example 8

| Component | Amount (mg) | % |
|---|---|---|
| Macitentan | 10.12 | 14.45 |
| Mannitol | 37.00 | 52.86 |
| Microcrystalline cellulose | 17.28 | 24.69 |
| Crospovidone | 2.10 | 3.00 |
| Povidone | 2.10 | 3.00 |
| Tween 80 | 0.70 | 1.00 |
| Magnesium stearate | 0.70 | 1.00 |
| Total | 70 | 100.00 |

A comparative compressed macitentan composition (see in table 9 the composition detail) was prepared according to the following process:
a) Macitentan, lactose monohydrate, povidone, microcrystalline cellulose, and sodium starch glycolate powders were mixed in appropriate equipment.
b) The mixture was granulated with a granulation solution prepared with polysorbate 80 and water. The granulate was dried at 40° C. and calibrated with a sieve aperture of a 1 mm.
c) The calibrated granulate was lubricated with magnesium stearate and compressed.

TABLE 9
Compressed macitentan composition (Comparative example)

| Component | Amount (mg) | % |
|---|---|---|
| Macitentan | 10.12 | 14.45 |
| Lactose monohydrate | 37.00 | 52.86 |
| Microcrystalline cellulose | 16.58 | 23.69 |
| Sodium starch glycolate | 2.10 | 3.00 |
| Povidone | 2.10 | 3.00 |
| Polysorbate 80 | 1.40 | 2.00 |
| Magnesium stearate | 0.70 | 1.00 |
| Total | 70 | 100.00 |

In an embodiment, the stability of the compositions was analysed. The compositions described in examples 1 to 4 and 7 and the composition of the comparative example were submitted to stress conditions in order to study the product behaviour and degradation profile throughout the time. The test batches were stored in closed bottles and left under two different conditions (a) 40° C. with 75% relative humidity, (b) 85% relative humidity. Each test batch was analysed after 1.5 months of exposition and/or after 3 months of exposition.

In an embodiment, each composition was analysed using a High Performance Liquid Chromatography system with a UV detector at 260 nm. For the samples analysis was used a validated and stability indicating methodology, and the quantification was performed using a characterized working standard. The MCT-IMA impurity, which is the prevalent impurity of Macitentan, and the total of impurities were quantified.

TABLE 10
Data related to the stability of the composition of the example 1-4 and
7, with SDS or polysorbate and maltitol, xylitol, mannitol, and the comparative example
(detailed on table 9) at 75% RH and 40° C.

| | Composition | | Composition | | Composition | | Composition | | Composition | | Comparative | |
|---|---|---|---|---|---|---|---|---|---|---|---|---|
| | Ex. 1 | | Ex. 2 | | Ex. 3 | | Ex. 4 | | Ex. 7 | | Example | |
| 40° C./75% RH | Time (months) | | | | | | | | | | | |
| Closed Bottle | 1.5 | 3 | 1.5 | 3 | 1.5 | 3 | 1.5 | 3 | 1.5 | 3 | 1.5 | 3 |
| MCT-IMA | 0.057 | 0.081 | 0.051 | 0.064 | 0.065 | 0.095 | 0.065 | 0.103 | 0.072 | — | 0.086 | 0.119 |
| impurity (%) | | | | | | | | | | | | |
| Total Impurities | 0.103 | 0.122 | — | 0.144 | 0.106 | 0.139 | 0.100 | 0.147 | 0.123 | — | 0.134 | 0.214 |
| (%) | | | | | | | | | | | | |

TABLE 11
Data related to the stability of the composition of the example 1-4 and
7, with SDS or polysorbate and maltitol, xylitol, mannitol, and the comparative example
(detailed on table 9) at 85% RH

| | Composition | Composition | Composition | Composition | Composition of | |
|---|---|---|---|---|---|---|
| | of Example | of Example | of Example | of Example | Example | Comparative |
| 85% RH | 1 | 2 | 3 | 4 | 7 | Example |
| Time | 3 | 3 | 3 | 3 | 3 | 3 |
| (months) | | | | | | |
| Total | 0.089 | 0.080 | 0.045 | 0.045 | 0.046 | 0.100 |
| Impurities | | | | | | |
| (%) | | | | | | |

In an embodiment, the compressed macitentan compositions of examples 1 to 4 and 7 exhibit enhanced stability as compared to the composition of the comparative example (detailed on table 9). Table 10 shows that after 3 months under 40° C. with 75% relative humidity, compositions of examples 1 to 4 and 7 exhibit lower MCT-IMA impurity level and lower total impurities level as compared to the composition of the comparative example. Table 11 shows that after 3 months under 85% relative humidity, compositions of examples 1 to 4 and 7 exhibit lower total impurities level than the composition of the comparative example.

In an embodiment, the stability of the compositions was analysed. The compositions described in examples 1, 2 and 5 and the composition of the comparative example were submitted to stress conditions in order to study the product behaviour and degradation profile throughout the time. The test batches were stored in closed bottles and left under 60° C. Each test batch was analysed twice, once after 1.5 months of exposition and once after 3 months of exposition.

In an embodiment, each composition was analysed using a High-Performance Liquid Chromatography system with a UV detector at 260 nm. For the samples analysis was used a validated and stability indicating methodology, and the quantification was performed using a characterized working standard. The MCT-IMA impurity, which is the prevalent impurity of Macitentan and the total of impurities were quantified.

TABLE 12
Data related to the stability of the composition of the example 1, 2, 5, with
SDS and xylitol, maltitol or mannitol, and the comparative example at 60° C.

| | | | | | | | Comparative | |
|---|---|---|---|---|---|---|---|---|
| | Composition 1 | | Composition 2 | | Composition 5 | | Example | |
| | Time (months) | | | | | | | |
| 60° C. | 1.5 | 3 | 1.5 | 3 | 1.5 | 3 | 1.5 | 3 |
| MCT-IMA | 0.402 | 0.601 | 0.114 | 0.426 | 1.184 | 1.733 | 2.867 | 5.752 |
| impurity | | | | | | | | |
| (%) | | | | | | | | |
| Total | 0.529 | 0.858 | 0.336 | 0.470 | 1.428 | 2.376 | 3.074 | 6.406 |
| Impurities | | | | | | | | |
| (%) | | | | | | | | |

Table 12 shows that at 60° C., the compressed macitentan compositions of Examples 1, 2 and 5 exhibit enhanced stability as compared to the composition of the comparative example (detailed on table 9). After 1.5 months and 3 months under 60° C., compositions of examples 1, 2 and 5 exhibit lower level of MCT-IMA impurity and lower level of total impurities than the composition of the comparative example.

TABLE 13
Data related to the stability of the composition
of the example 6, at 40° C. and 30° C.

| Conditions | 40° C./75% HR | | 30° C./75% HR | |
|---|---|---|---|---|
| Time (months) | 6 | 12 | 6 | 12 |
| MCT-IMA impurity (%) | <0.05% | n/a | <0.05% | <0.05% |
| Total Impurities (%) | <0.05% | n/a | <0.05% | <0.05% |

Table 13 shows that at 40° C. or 30° C., the compressed macitentan compositions of Examples 6 exhibit enhanced stability as compared to the composition of the comparative example (detailed on table 9).

A dissolution test was also performed. In the dissolution test of the disclosed examples were performed in 900 mL of dissolution medium at 37° C. 0.5° C., using USP Apparatus 2 (paddles) method at a rotation speed of 50 rpm. Samples are removed after 5, 10, 20, 30, 45, 60 and 90 minutes from test initiation and analyzed for dissolved macitentan using a suitable HPLC method at 260 nm. pH 6.8 phosphate buffer+0.1% CTAB had been used as dissolution medium, with adequate discrimination ability. The reported dissolution results are average values from six tablets.

Table 14 shows the dissolution profiles of the example compositions as compared with the reference drug product (Opsumit®). The dissolution tests were performed under same conditions, using a similarity factor (f2). The similarity factor (f2) is a measurement of the similarity in percentage of dissolution between two curves. Two dissolution profiles are considered similar when the f2 value is ≥50.

TABLE 14
Data related to the dissolution profiles of an oral solid composition of
the example 1, 2, 3 ,5 and the composition of the Opsumit ® tablets

| f2(%) | Example 1 | Example 2 | Example 3 | Example 5 |
|---|---|---|---|---|
| Opsumit ® | 59 | 66 | 77 | 73 |

The compressed macitentan compositions tested showed good similarity when compared to commercial solid oral compositions comprising macitentan (Opsumit® tablets).

TABLE 15
Data related to the dissolution profiles of an oral
solid composition of the example 6 in 0.1 N HCl

| Time (min) | % |
|---|---|
| 0 | 0 |
| 5 | 48 |
| 10 | 67 |
| 15 | 74 |
| 30 | 84 |
| 45 | 88 |
| 60 | 90 |
| 90 | 92 |

TABLE 16
Data related to the dissolution profiles of an oral solid composition of
the example 6 in phosphate buffer, pH 6.8, 0.02% CTAB and 50 rpm

| Time | | (30° C./75% HR) after | (40° C./75% HR) after |
|---|---|---|---|
| (min) | T0 | 6 months storage | 6 months storage |
| 0 | 0 | 0 | 0 |
| 5 | 42 | 40 | 38 |
| 10 | 60 | 58 | 61 |
| 15 | 67 | 66 | 71 |
| 30 | 79 | 77 | 85 |
| 45 | 85 | 82 | 91 |
| 60 | 89 | 86 | 95 |
| 90 | 93 | 90 | 99 |

TABLE 17
Data related to the dissolution profiles of an oral solid composition of
the example 6, 7 and 8 in phosphate buffer pH 6.8, 0.1% CTAB and 50 rpm

| | Example 6 | Example 7 | Example 8 |
|---|---|---|---|
| Time (min) | T0 | T0 | T0 |
| 0 | 0 | 0 | 0 |
| 5 | 35 | 22 | 24 |
| 10 | 51 | 50 | 50 |
| 15 | 60 | 65 | 62 |
| 30 | 74 | 83 | 77 |
| 45 | 81 | 92 | 85 |
| 60 | 84 | 98 | 89 |
| 90 | 91 | 100 | 94 |

TABLE 18
Data related to the dissolution profiles of an oral solid composition of
the example 6, 7 and 8 and the composition of the Opsumit ® tablets

| f2(%) | Example 6 | Example 7 | Example 8 |
|---|---|---|---|
| Opsumit ® | 60 | 68 | 62 |

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The term “comprising” whenever used in this document is intended to indicate the presence of stated features, integers, steps, components, but not to preclude the presence or addition of one or more other features, integers, steps, components or groups thereof.

The above described embodiments are combinable.

1. A compressed composition, comprising:
a therapeutically effective amount of macitentan;
a sugar alcohol diluent selected from the group consisting of: maltitol, xylitol, mannitol, and mixtures thereof; and
a surfactant selected from the group consisting of: sodium dodecyl sulfate, polysorbate 80, and a mixture thereof.

2. The compressed composition claim 1, comprising 0.1% to 15% (wt_{surfactant}/wtₜₒₜₐₗ) of the surfactant.

3. The compressed composition claim 2, comprising 0.5% to 15% (wt_{surfactant}/wtₜₒₜₐₗ) of the surfactant.

4. The compressed composition of claim 1, comprising from 0.9% to 10% (wt_{surfactant}/wtₜₒₜₐₗ) of the surfactant.

5. The compressed composition of claim 1, comprising from 0.5 to 2% (wt_{surfactant}/wtₜₒₜₐₗ) of the surfactant.

6. The compressed composition of claim 1, comprising:
maltitol as the sugar alcohol diluent and sodium dodecyl sulfate as the surfactant; or
maltitol as the sugar alcohol diluent and polysorbate 80 as the surfactant.

7. The compressed composition of claim 1, further comprising: 5% to 65% (wt_{non-sugar alcohol diluent}/wtₜₒₜₐₗ) of a non-sugar alcohol diluent.

8. The compressed composition of claim 7, comprising microcrystalline cellulose as the non-sugar alcohol diluent.

9. The compressed composition of claim 1, comprising from 15% to 85% (wt_{sugar alcohol diluent}/wtₜₒₜₐₗ) of the sugar alcohol diluent.

10. The compressed composition of claim 1, comprising from 1% to 45% (wt_{macitentan}/wtₜₒₜₐₗ) of macitentan.

11. The compressed composition of claim 1, further comprising: from 10% to 50% (wt_{non-sugar alcohol diluent}/wtₜₒₜₐₗ) of a non-sugar alcohol diluent.

12. The compressed composition of claim 1, wherein the compressed composition comprises from 5% to 25% (wt_{macitentan}/wtₜₒₜₐₗ) of macitentan, from 35% to 65% (wt_{sugar alcohol diluent}/wtₜₒₜₐₗ) of the sugar alcohol diluent, and from 0.5% to 5% (wt_{surfactant}/wtₜₒₜₐₗ) of the surfactant.

13. The compressed composition of claim 1, further comprising at least one excipient selected from a disintegrant, a binder or a lubricant.

14. The compressed composition of claim 13, further comprising a non-sugar alcohol diluent, wherein the non-sugar alcohol diluent is microcrystalline cellulose, the disintegrant is crospovidone or sodium starch glycolate, the binder is povidone, and the lubricant is magnesium stearate.

15. The compressed composition of claim 1, wherein the composition is prepared by wet granulation.

16. The compressed composition of claim 1, wherein the composition is suitable for the treatment of pulmonary arterial hypertension.

17. A process for preparing a compressed composition, or a solid oral pharmaceutical composition, or a solid oral pharmaceutical composition suitable for the treatment of pulmonary arterial hypertension, comprising:
mixing macitentan, a sugar alcohol diluent, and optionally one or more excipients selected from a non-sugar alcohol diluent, a disintegrant, a binder, and a glidant, to form a mixture;
granulating the mixture with an aqueous solution of a surfactant wherein the surfactant is sodium dodecyl sulfate, or polysorbate 80, or a combination thereof, to form a granulate; and
lubricating the granulate; and
compressing the lubricated granulate.

18. A solid oral pharmaceutical composition comprising the compressed composition of claim 1, wherein the composition is suitable for the treatment of pulmonary arterial hypertension.

19. The compressed composition of claim 1, comprising:
xylitol as the sugar alcohol diluent and sodium dodecyl sulfate as the surfactant; or
xylitol as the sugar alcohol diluent and polysorbate 80 as the surfactant.

20. The compressed composition of claim 1, comprising:
mannitol as the sugar alcohol diluent and sodium dodecyl sulfate as the surfactant; or
mannitol as the sugar alcohol diluent and polysorbate 80 as the surfactant.
